(19) United States
(12) Patent Application Publication
(10) Pub. No.: US 2011/0033529 A1
(43) Pub. Date: Feb. 10, 2011

(54) ORAL PHARMACEUTICAL PARICALCITOL FORMULATIONS

(76) Inventors: Durga Prasad Samantaray, Khurda (IN); V. Nageswara Rao, Srikakulam (IN); Ravi Kumar Namballa, Hyderabad (IN); Bhushan Subhash Yeola, Nasik (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/578,700

(22) Filed: Oct. 14, 2009

(30) Foreign Application Priority Data
Aug. 6, 2009 (IN) 1872/CHE/2009

(51) Int. Cl. A61K 9/48; A61K 31/047; A61P 5/18

(52) U.S. Cl. 424/456; 514/729

(57) ABSTRACT

Pharmaceutically acceptable liquid paricalcitol formulations intended for oral administration, processes for preparing such formulations, and methods of using the same. Embodiments relate to liquid paricalcitol formulations filled into gelatin capsules.

### INTRODUCTION

Aspects of the present invention relate to paricalcitol pharmaceutical formulations intended for oral administration, processes for preparing such formulations, and methods of using the same. In embodiments, the present invention relates to liquid paricalcitol formulations filled into gelatin capsules.

The drug compound having the adopted name “paricalcitol” is a synthetically manufactured analog of calcitriol, the metabolically active form of vitamin D indicated for the prevention and treatment of secondary hyperparathyroidism in chronic kidney disease. A chemical name for paricalcitol is 19-nor-1α,3β,25-trihydroxy-9,10-secoergosta-5(Z),7(E),22(E)-triene, corresponding to the following structural Formula I.

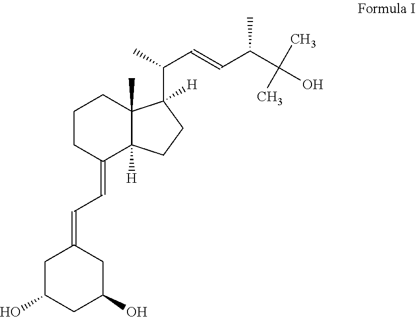

A commercially available oral Paricalcitol product is soft gelatin capsules sold as ZEMPLAR® capsules, distributed by Abbott. ZEMPLAR products are available in the form of soft gelatin capsules for oral administration containing 1 μg, 2 μg, or 4 μg of paricalcitol. ZEMPLAR capsules are indicated for the prevention and treatment of secondary hyperparathyroidism in chronic kidney disease.

U.S. Pat. No. 5,587,497 discloses vitamin D-related compounds, namely the 1α-hydroxy-19-nor-vitamin D analogs including paricalcitol, as well as a general method for their chemical synthesis.

U.S. Pat. No. 6,136,799 discloses an aqueous pharmaceutical formulation for parenteral administration having paricalcitol, about 50% (v/v) of an organic solvent, and about 50% (v/v) water.

U.S. Patent Application Publication No. 2006/0009425 discloses oral formulations of paricalcitol dissolved in a non-polar solvent in a particular ratio. U.S. Patent Application Publication No. 2006/0030549 discloses oral formulations with paricalcitol dissolved in a non-polar solvent in a particular ratio.

The product ZEMPLAR® is available as soft gelatin capsules for oral administration. Though the soft gelatin capsule is a conventional product, it has been observed that commercial scale manufacturing of soft gel products has certain problems. The drawbacks associated with soft gelatin dosages includes, but are not limited to, complexity of the equipment, high cost involved, a large dimensional product variation leading to problem in primary packaging, migration and degradation of sensitive drugs, especially those that are readily soluble in glycerol, migration of a drug into the soft gelatin capsule shell resulting in drug degradation, high moisture and oxygen permeability of the soft gel, and the comparatively high processing times.

Further the soft gelatin capsule dosage forms are not suitable for small batches, particularly due to the associated high gelatin waste that adds to the cost of the product manufacture. It also needs relatively low and controlled temperature and humidity conditions during manufacturing and drying operations, which further add to the overhead cost of making the product.

However, soft gelatin capsules are preferred for making formulations wherein a better aesthetic appeal is desired.

Further, since the paricalcitol formulations known to the art are in the form of soft gelatin capsules particularly comprising a non-polar excipient/oily substance in a relatively high proportion; such formulations exhibit a decreased dissolution rate/drug release and sometimes instability particularly during the shelf life of the product.

### SUMMARY

Aspects of the present invention relate to liquid paricalcitol formulations intended for oral administration in the form of a capsule.

Aspects of the present invention relate to pharmaceutically acceptable paricalcitol liquid formulations in the form of a hard gelatin capsule.

In an aspect, the present invention provides pharmaceutically acceptable paricalcitol liquid formulations in the form of a hard gelatin capsule with banding.

In another aspect, the present invention provides pharmaceutically acceptable paricalcitol liquid formulations for oral administration, comprising at least one solubilizer and at least one other pharmaceutically acceptable excipient.

In yet another aspect, the invention provides pharmaceutically acceptable paricalcitol liquid formulations for oral administration, comprising at least one solubilizer and at least one other pharmaceutically acceptable excipient, filled into a hard gelatin capsule with banding.

In another aspect, the present invention includes oral formulations comprising paricalcitol in a variety of dosage strengths that are “proportionally similar”.

The present invention also relates to process for preparing liquid paricalcitol formulations intended for oral administration.

In a further aspect, the invention includes methods for prevention and treatment of secondary hyperparathyroidism associated with chronic kidney disease (CKD) Stage 3 and 4, and CKD Stage 5 patients on hemodialysis (HD) or peritoneal dialysis (PD). using pharmaceutical formulations of the present invention.

### DETAILED DESCRIPTION

Aspects of the present invention relate to pharmaceutically acceptable paricalcitol liquid formulations for oral administration. In embodiments, the formulations are in the form of hard gelatin capsules with banding.

In an aspect, the present invention provides pharmaceutically acceptable paricalcitol liquid formulations for oral administration, comprising at least one solubilizer along with one or more other pharmaceutically acceptable excipients.

The term “paricalcitol” as used herein refers to the compound, and to any one or more of its salts, polymorphs, solvates, esters, hydrates, enantiomers, racemates, and mixtures thereof.

The term “liquid” as used herein refers to one or more of a solution, suspension, emulsion, or colloidal dispersion. The active agent, i.e., paricalcitol, may be in a dissolved state, a dispersed state, or a combination of both, in a liquid matrix. Average particle sizes of the active agent, if in a dispersed state, vary from about 0.1 nm to about 100 μm, or more. Average particle sizes of the active agent used to make the liquid formulation of the present invention can be in the range of about 0.5 μm to about 500 μm, such as are measured using a light scattering technique.

The solubilizers are chosen to be compatible with a gelatin capsule shell and the active agent paricalcitol. In certain embodiments, they dissolve paricalcitol and yet do not substantially hydrolyze, dissolve, or discolor the capsule shell. Embodiments of the invention include use of one or more polyoxyethylated castor oils (e.g., CREMOPHOR® RH 40 and CREMOPHOR® ELP, from BASF) as a solubilizer.

CREMOPHOR ELP is generally known as a surfactant in the pharmaceutical industry and plays an important role in emulsification or solubilization of fat-soluble vitamins A, D, E, and K in aqueous solutions, for oral and topical administration. It also contributes to oral bioavailability enhancement of these vitamins.

In an aspect, it was found by the present inventors that when CREMOPHOR ELP is used as a solubilizer, it also contributes in decreasing the inter-individual pharmacokinetic variations, particularly the variability with respect to drug plasma levels (AUC and Cₘₐₓ).

The term “solubilizer” as used herein is synonymous with “surfactant,” “wetting agent.” and “emulsifying agent” and include agents which are used to solubilize the drug in a particular solvent.

Other suitable solubilizers that can be used in the present invention include surfactants that may be ionic or nonionic. Ionic surfactants may be anionic, cationic, or zwitterionic. Anionic surfactants include the alkoyl isethionates, alkyl and alkyl ether sulfates and salts thereof, alkyl and alkyl ether phosphates and salts thereof, alkyl methyl taurates, and soaps, such as, for example, alkali metal salts including sodium or potassium salts of long chain fatty acids. Non-limiting examples include chenodeoxycholic acid, 1-octanesulfonic acid sodium salt, sodium deoxycholate, glycodeoxycholic acid sodium salt, N-lauroylsarcosine sodium salt, lithium dodecyl sulfate, sodium cholate hydrate, and sodium lauryl sulfate (SLS), also called sodium dodecyl sulfate (SDS).

Examples of amphoteric and zwitterionic surfactants include, but are not limited to, carboxy, sulfonate, sulfate, phosphate, and phosphonate compounds. Examples are alkylimino acetates and iminodialkanoates and aminoalkanoates, imidazolinium and ammonium derivatives, betaines, sultaines, hydroxysultaines, alkyl sarcosinates and alkanoyl sarcosinates, and the like.

Nonionic surfactants include polyoxyethylene castor oil derivatives, polyoxyethylene sorbitan fatty acid esters (e.g., the commercially available TWEEN® products, including TWEEN 20 and TWEEN 800, from ICI Specialty Chemicals), poloxamers (e.g., PLURONIC® products F68, F127 and F108Q, which are block copolymers of ethylene oxide and propylene oxide, from BASF Corporation), poloxamines (e.g., TETRONIC® 908, also known as poloxamine 908, which is a tetrafunctional block copolymer derived from sequential addition of propylene oxide and ethylene oxide to ethylenediamine, from BASF Wyandotte Corporation, Parsippany, N.J. USA), TETRONIC 15080 (T-1508) (BASF Wyandotte Corporation), and D-alpha tocopheryl polyethylene glycol 1000 succinate (e.g., the commercially available SPEZIOL® TPGS Pharma, supplied by Cognis GmbH).

Examples of useful cationic surfactants include, but are not limited to, polymers, biopolymers, polysaccharides, cellulosics, alginates, phospholipids, and nonpolymeric compounds, such as zwitterionic stabilizers, poly-n-methylpyridinium, anthrylpyridinium chloride, cationic phospholipids, chitosan, polylysine, polyvinylimidazole, polybrene, polymethylmethacrylate, polyvinylpyrrolidone-2-dimethylaminoethyl methacrylate, lysozyme, long-chain polymers such as alginic acid, carrageenan (FMC Corp.), and POLYOX™ (Dow Chemical Co., Midland, Mich. USA), cationic lipids, sulfonium, phosphonium, and quarternary ammonium compounds, such as stearyltrimethylammonium chloride, and benzyl-di-(2-chloroethyl)ethylammonium bromide.

Other pharmaceutically acceptable excipients that can be present in the liquid formulations include one or more of non-polar solvents, polar solvents, medium polar solvents, neutral solvents, co-solvents, surfactants, antioxidants, low molecular weight alcohols, preservatives, buffering agents, and the like. One or more of these excipients are frequently present in the liquid formulations comprising paricalcitol.

Useful non-polar solvents comprise, without limitation thereto, short chain aliphatic or aromatic hydrocarbons, alkyl-substituted solvents, medium chain triglycerides and mixtures thereof.

The non-polar solvent chosen for use in the present invention should not react detrimentally with, or cause degradation of, the paricalcitol. Suitable non-polar solvents include, but are not limited to, short chain aliphatic or aromatic hydrocarbons, alkyl-substituted solvents, medium chain triglycerides, and mixtures thereof. The non-polar solvents can contain between 2 to 14 carbon atoms in a carbon chain, and may contain multiple carbon chains.

In embodiments, non-polar solvents are medium chain triglycerides containing between 6 and 12 carbon atoms per carbon chain. Examples of non-polar solvents that can be used in the present invention, include, but are not limited to, caprylic/capric triglyceride (i.e., NEOBEE® M-5, from Stepan Company, Northfield, Ill., USA.), canola oil, corn oil, cottonseed oil, ethyl oleate, isopropyl myristate, isopropyl palmitate, light mineral oil, mineral oil, peanut oil, and soybean oil.

Suitable polar solvents also include one or more of low molecular weight pharmacologically acceptable alcohols, water, glycerin, polyethylene glycols, hexanes, propylene glycols, and the like. In an aspect, the amount of polar solvent used in the formulation is such that it is sufficient to solubilize the active agent paricalcitol, and wherein such quantity does not cause any detrimental effect on the capsule shell. Excess amounts of solvent can sometimes be detrimental. In an aspect, the quantity of polar solvent present in the liquid formulation is such that it is in equilibrium with an amount of polar solvent present in the capsule shell so as to prevent or at least minimize the migration between the liquid fill material and the capsule shell.

In an aspect, a polar or neutral solvent may also be used in combination with a non-polar solvent. Weight ratios of polar or neutral solvent to non-polar solvent can be in the range of about 1:100 to 100:1.

Aspects of the present invention further extend to oral formulations comprising paricalcitol dissolved in a neutral solvent, with or without one or more other pharmaceutically acceptable excipients.

Suitable neutral solvents comprise one or more of polysorbate 80, polyoxyl castor oil (CREMOPHOR RH 40 and CREMOPHOR, ELP from BASF), and any mixtures thereof.

A polar or neutral solvent is chosen such that it is compatible with the gelatin shell and paricalcitol. The solvent usually dissolves paricalcitol and yet not does hydrolyze, dissolve, or discolor the capsule shell. However, “other components” wherein the paricalcitol is not appreciably soluble or is only slightly soluble may be used and such other components include, but are not limited to, cyclodextrins, LABRASOL®, GELUCIRE® 44/14 or 50/13, and the like. The other component may include but is not limited to any other emulsifiers used to enhance solubility of paricalcitol.

The antioxidants that can be used include, but are not limited to, butylated hydroxytoluene, alpha tocopherol, ascorbic acid, ascorbyl palmitate, butylated hydroxyanisole, propyl gallate, sodium ascorbate, sodium metabisulfite, and mixtures thereof.

A ‘low molecular weight alcohol’ refers to an aliphatic alcohol having from 1 to about 5 carbons, such as, for example, ethanol, propanol, butanol, and/or mixtures thereof.

In an embodiment, the present invention also provides oral formulations comprising paricalcitol dissolved in a non-polar solvent, with or without other pharmaceutically acceptable excipients.

The capsules used for the filling the liquid formulation may be a hard gelatin capsules or soft gelatin capsules. In embodiments, hard gelatin capsules are used. A hard gelatin capsule has gelatin and a water dispersible or water-soluble plasticizer as principal components, and may contain other suitable additives such as preservatives, coloring agents, and/or opacifiers that are present to stabilize the capsule and/or impart a specific characteristic such as color or other appearance attribute to the capsule. The hard capsules are usually pre-fabricated and supplied empty. In comparison to soft gelatin capsules, the moisture uptake of hard gelatin capsules is usually much lower, facilitating processing. However, soft gelatin capsules are desirable when an aesthetic appeal is intended to be provided to the product. A soft gelatin capsule is a one-piece, hermetically sealed soft gelatin shell containing a liquid, a suspension or a semisolid filled therein. The soft gelatin shell usually comprises a film-forming material such as gelatin, and a water dispersible or water-soluble plasticizer to impart flexibility. The soft gelatin shell may also include additives such as coloring agents, flavors, sweeteners and preservatives.

In an aspect, the formulations of the present invention are bio-equivalent to the commercially available oral paricalcitol product in the form of a soft gelatin capsule, sold under the brand name ZEMPLAR®, from Abbott Laboratories.

The present invention includes oral formulations comprising paricalcitol that are prepared in a variety of dosage strengths which are bioequivalent to one another and are “proportionally similar.”

More specifically, the present invention describes dosage forms in which the active and inactive ingredients are varied proportionately (i.e., to make them proportionally similar) in order to prepare bioequivalent products having different dose strengths. In an aspect, the invention also describes dosage form formulations in which the active and inactive ingredients are varied “pseudo-proportionately” (i.e., to make them pseudo-proportionally similar).

The oral formulations of the present invention can be prepared in a variety of dosage strengths such as, for example. where the quantity of paricalcitol in said formulations is 0.25 μg, 0.5 μg, 1 μg, 2 μg, 4 μg, 8 μg, 16 μg, and the like, that are bioequivalent, when dosed at the same total weight of paricalcitol, to each other, and are “proportionally similar” or “pseudo-proportionately similar”.

The term “proportionally similar” for a potent drug, i.e., paricalcitol, according to Definition 3 of the U.S. Food and Drug Administration guidance for “Bioavailability and Bioequivalence Studies for Orally Administered Drug Products—General Considerations” (March 2003) is: “where the amount of the active drug substance in the dosage form is relatively low, the total weight of the dosage form remains nearly the same for all strengths (within ±10% of the total weight of the strength on which a bio-study is performed), the same inactive ingredients are used for all strengths, and the change in any strength is obtained by altering the amount of the active ingredients and one or more of the inactive ingredients. The changes in the inactive ingredients are within the limits defined by the SUPAC-IR and SUPAC-MR guidance up to and including Level II.”

The term “pseudo-proportionately similar” for a potent drug, i.e., paricalcitol, refers to the formulations in which the different dosage strengths are substantially proportional. The term ‘substantially proportional’ implies that the percentage weight difference between two dosage strengths is less than about 10%, or less than about 5%, or less than about 1%.

The present invention also provides processes for preparing liquid paricalcitol formulations intended for oral administration. In an aspect, processes of preparation of the liquid formulations comprise combining the active agent with at least one solubilizer, optionally together with one or more pharmaceutically acceptable excipients, and filling the liquid into a hard gelatin capsule or encapsulating within a soft gelatin capsule.

In an embodiment, the hard gelatin capsules are banded to prevent leakage. Various methods can be used to band the hard gelatin capsules which include, but are not limited to, banding using a gelatin band and sealing using a hydroalcoholic solution.

In an embodiment of the present invention, a method wherein the capsules are filled and then sealed by spraying a small amount of a water-ethanol mixture at the cap and body interface, followed by warming to fuse the two capsule parts together, is used. The banding of hard gelatin capsules is well-known in the art. The capsules are first rectified and then passed once or twice over a wheel that revolves in a gelatin bath. An amount of gelatin is picked up by the serrated wheel and applied to the junction of the cap and body. The capsules remain in individual carriers for drying. Instrumentation for performing the encapsulation, banding, and sealing are commercially available, for example, the FLUIDOCAP™ F-40, FLUIDOCAP S-40 and PAM 1000 SA LLF machines from PAM USA LLC; and the HICAPSEAL™ (S-40 and S-100) capsule sealing equipment from Qualicaps Group.

The liquid formulations of the present invention are highly stable during the manufacture as well as upon storage under commercially relevant conditions, for a commercially acceptable time. In general, pharmaceutical products are expected to be stored at about room temperature, for periods of six months, one year, two years, or any intermediate times.

Embodiments of stability include one or more of the following: the finished product contains not more than about 3% of any identified or unidentified individual drug-related impurity, or not more than about 1%; and the total drug-related impurities in the finished product are not more than about 5%, or not more than about 3%; by weight of the label amount of active agent present.

In some embodiments, the invention includes use of packaging materials such as containers and closures of high-density polyethylene (HDPE), low-density polyethylene (LDPE) and or polypropylene and/or glass, glassine foil, aluminum pouches, and blisters or strips composed of aluminum or high-density polyvinyl chloride (PVC), optionally including a desiccant, polyethylene (PE), polyvinylidene dichloride (PVDC), PVC/PE/PVDC, and the like.

In embodiments, the packaging comprises a high-density polyethylene (HDPE) container with a child resistant cap (CRC), sealed by induction sealing.

In a further aspect, the invention includes methods for prevention and/or treatment of secondary hyperparathyroidism associated with chronic kidney disease (CKD) Stage 3 and 4, and CKD Stage 5 patients on hemodialysis (HD) or peritoneal dialysis (PD) using pharmaceutical formulations of the present invention.

The following examples illustrate certain specific aspects and embodiments of the invention and demonstrate the practice and advantages thereof. It is to be understood that the examples are provided solely for purposes of illustration and are not intended to limit the scope of the invention in any manner.

### Example 1

### Paricalcitol Capsules

| | Quantity per | |
|---|---|---|
| Ingredient | Capsule | |
| Paricalcitol | 4 | μg |
| Dehydrated alcohol | 2.84 | mg |
| Butylated hydroxytoluene | 16 | μg |
| CREMOPHOR ® ELP** | 1.42 | mg |
| NEOBEE M-5\hat{ } | 137.72 | mg |

**CREMOPHOR ® ELP is a nonionic solubilizer comprising polyoxyethylated castor oil, made by reacting castor oil with ethylene oxide in a molar ratio of 1:35 followed by a purification process, and is supplied by BASF Aktiengesellschaft.
\hat{ }NEOBEE M-5 comprises caprylic/capric triglyceride, and is supplied by Stepan Company, Northfield, Illinois.

Manufacturing Process:

1. Dissolve butylated hydroxytoluene in dehydrated alcohol.

2. Dissolve paricalcitol in the solution of step 1.

3. Mix the materials of steps 1 and 2.

4. Add CREMOPHOR ELP to the solution of step 3 and mix to get a clear solution.

5. Add the solution of step 4 to NEOBEE M-5 and mix to obtain a homogeneous solution.

6. Fill into hard gelatin capsules.

7. Band seal the capsules with gelatin mass, as follows:
a) Rectify the filled capsule of step 6 on a banding machine.
b) Pass the capsules at least twice over a wheel that revolves in a gelatin bath.
c) Dry the band sealed capsules.

The formulation of Example 1 and ZEMPLAR® 4 μg capsules are subjected to dissolution testing using 500 mL of 0.4% lauryldimethylamine N-oxide (LDAO) in purified water, 100 RPM stirring, using a USP Type I (basket) apparatus. The cumulative percentages of drug dissolved are tabulated in Table 1.

TABLE 1

| Minutes | Example 1 | ZEMPLAR |
|---|---|---|
| 20 | 92 | 83 |
| 30 | 100 | 95 |
| 45 | 101 | 104 |
| 60 | 103 | 108 |

Tablets prepared in Example 1 are evaluated in an open label, balanced, randomized two treatment, two-sequence, two period, two way crossover, single dose comparative bioavailability study with administration of the test product and the commercial product Zemplar® 4 μg capsules, to 16 fasting healthy human subjects, with a washout period of 7 to 21 days. Calculated pharmacokinetic parameter mean values are given in Table 2.

TABLE 2

| | AUC₍₀₋ₜ₎ | AUC_{(0-∞)} | Cₘₐₓ |
|---|---|---|---|
| | (pg · hour/mL) | (pg · hour/mL) | (pg/mL) |
| Example 1 (“T”) | 1967.83 | 2455.478 | 169.763 |
| ZEMPLAR (“R”) | 2002.371 | 2479.778 | 180.65 |
| Least Square Mean | 102.10 | 101.26 | 106.83 |
| (T ÷ R × 100) | | | |

### Example 2

### Paricalcitol Capsules

Formulation is similar to that of Example 1.

Manufacturing process for filling capsules is similar to that of Example 1.

Banding is accomplished using LEMS™ technology:
a) Spray a 50:50 by volume water-ethanol mixture onto the joint between capsule halves.
b) Apply gentle heat of about 45° C. and fuse together to form a complete 360° seal.
c) Maintain the capsule at room temperature to harden the seal.

### Example 3

### Paricalcitol Capsules

| | Quantity per | |
|---|---|---|
| Ingredient | Capsule | |
| Paricalcitol | 4 | μg |
| Dehydrated alcohol | 2 | mg |
| Butylated hydroxytoluene | 16 | μg |
| NEOBEE M-5 | 137.98 | mg |

Manufacturing Process:

1. Dissolve butylated hydroxytoluene in dehydrated alcohol.

2. Dissolve paricalcitol in the solution of step 1.

3. Mix the solution of step 2 with NEOBEE M-5 to obtain a homogeneous solution.

4. Fill into hard gelatin capsules.

5. Band seal the capsules with gelatin mass, using the process of Example 1, step 7.

A fasting state pharmacokinetic study is conducted with 16 subjects, administering the formulations of Example 1 and Example 3. Calculated mean pharmacokinetic parameters are given in Table 3.

TABLE 3

| | AUC₍₀₋ₜ₎ | AUC_{(0-∞)} | Cₘₐₓ |
|---|---|---|---|
| Formulation | (pg · hour/mL) | (pg · hour/mL) | (pg/mL) |
| Example 3 | 1244.454 | 1438.58 | 143.855 |
| | (% CV = 31.8) | (% CV = 30.9) | (% CV = 23.4) |
| Example 1 | 1967.83 | 2455.478 | 169.763 |
| | (% CV = 12.7) | (% CV = 14.5) | (% CV = 11.8) |

The data in Table 3 indicate that the inter-individual variations in AUC and Cₘₐₓ are less pronounced for Example 1 (with CREMOPHOR ELP) than for Example 3 (without CREMOPHOR ELP). This was judged by the lower values for coefficient of variation (% CV) for the AUC and Cₘₐₓ.

### Example 4

### Paricalcitol Capsules

| | mg per Capsule | | |
|---|---|---|---|
| | 1 μg | 2 μg | 4 μg |
| Ingredient | Strength | Strength | Strength |
| Paricalcitol | 0.001 | 0.002 | 0.004 |
| Butylated hydroxytoluene | 0.008 | 0.016 | 0.032 |
| Ethanol | 1.375 | 2.75 | 5.5 |
| Super refined olive oil | 154.616 | 309.232 | 618.464 |

Manufacturing Process:

1. Dissolve butylated hydroxytoluene in ethanol.

2. Add paricalcitol to the solution of step 1.

3. Dissolve Super refined olive oil in the step 2 solution, and mix to obtain homogeneity.

4. Fill the mixture from step 3 into a capsule.

### Example 5

### Paricalcitol Capsules

| | 1 μg Strength | | 2 μg Strength | | 4 μg Strength | |
|---|---|---|---|---|---|---|
| Ingredient | mg | Wt. % | mg | Wt. % | mg | Wt. % |
| Paricalcitol | 0.001 | 0.0007 | 0.002 | 0.0014 | 0.004 | 0.0028 |
| Butylated hydroxytoluene | 0.016 | 0.0114 | 0.016 | 0.0114 | 0.016 | 0.0114 |
| Absolute ethanol | 1.483 | 1.059 | 1.482 | 1.058 | 1.48 | 1.057 |
| NEOBEE M-5 | 138.5 | 98.93 | 138.5 | 98.93 | 138.5 | 98.93 |
| Total | 140 | 100 | 140 | 100 | 140 | 100 |

Manufacturing process is similar to that described in Example 4.

Difference in Wt. % Values

| | 4 μg Strength − | 4 μg Strength − |
|---|---|---|
| Ingredient | 1 μg Strength | 2 μg Strength |
| Paricalcitol | 0.0021 | 0.0014 |
| Butylated hydroxytoluene | 0 | 0 |
| Absolute ethanol | 0.002 | 0.001 |
| NEOBEE M-5 | 0 | 0 |
| Total | 0.004 | 0.002 |

The component percentage differences between the 1 μg and 4 μg dosage strengths, and between the 2 μg and 4 μg dosage strengths, are very small. Hence the fill formulations for 1 μg, 2 μg, and 4 μg are “proportionally similar” or “pseudo-proportionally similar” according to Definition 3 of the U.S. Food and Drug Administration guidance for “Bioavailability and Bioequivalence Studies for Orally Administered Drug Products—General Considerations” (March 2003).

### Example 6

### Paricalcitol Capsules

| | mg per Capsule | | |
|---|---|---|---|
| | 1 μg | 2 μg | 4 μg |
| Ingredient | Strength | Strength | Strength |
| Paricalcitol | 0.001 | 0.002 | 0.004 |
| Dehydrated alcohol | 1 | 2 | 4 |
| Propylene glycol | 138.999 | 277.998 | 555.996 |

Manufacturing Process:

1. Dissolve paricalcitol in dehydrated alcohol.

2. Add propylene glycol to the solution of step 1 and mix to obtain homogeneity.

3. Fill the mixture from step 2 into a capsule.

### Example 7

### Paricalcitol Capsules

| | mg per Capsule | | |
|---|---|---|---|
| | 1 μg | 2 μg | 4 μg |
| Ingredient | Strength | Strength | Strength |
| Paricalcitol | 0.001 | 0.002 | 0.004 |
| Dehydrated alcohol | 2 | 2 | 2 |
| Polyethylene glycol 400 | 137.999 | 137.998 | 137.996 |

Manufacturing Process:

1. Dissolve paricalcitol in dehydrated alcohol.

2. Add propylene glycol to the solution of step 1 and mix to obtain homogeneity.

3. Fill the mixture of step 2 into a capsule.

### Example 8

### Paricalcitol Capsules

| | mg per Capsule | | |
|---|---|---|---|
| | 1 μg | 2 μg | 4 μg |
| Ingredient | Strength | Strength | Strength |
| Paricalcitol | 0.001 | 0.002 | 0.004 |
| Butylated hydroxytoluene | 0.008 | 0.008 | 0.008 |
| Ethanol | 1.375 | 1.375 | 1.375 |
| GELUCIRE ® 44/14# | q.s. | q.s. | q.s. |
| Polysorbate 80 | 146.616 | 146.614 | 146.612 |

#GELUCIRE ® 44/14 comprises lauroyl macrogolglycerides and is supplied by Gattefosse, St-Priest France.

Manufacturing Process:

1. Dissolve butylated hydroxytoluene in ethanol.

2. Add paricalcitol to the solution of step 1.

3. Dissolve polysorbate 80 and GELUCIRE® 44/14 in the step 2 mixture, and mix to obtain homogeneity.

4. Fill the mixture of step 3 into a capsule.

### Example 9

### Paricalcitol Capsules

| | mg per Capsule | | |
|---|---|---|---|
| | 1 μg | 2 μg | 4 μg |
| Ingredient | Strength | Strength | Strength |
| Paricalcitol | 0.001 | 0.002 | 0.004 |
| Butylated hydroxytoluene | 0.008 | 0.008 | 0.008 |
| Ethanol | 1.375 | 1.375 | 1.375 |
| Sesame oil | 146.616 | 146.614 | 146.612 |

Manufacturing Process:

1. Dissolve paricalcitol in ethanol.

2. Add butylated hydroxytoluene to the solution of step 1.

3. Add sesame oil to the step 2 mixture and mix to obtain homogeneity.

4. Fill the mixture of step 3 into a capsule.

We claim:

1. A liquid formulation for oral administration, comprising paricalcitol, at least one solubilizer, and one or more other pharmaceutically acceptable excipients, present in a capsule.

2. The liquid formulation according to claim 1, being present in a hard gelatin capsule.

3. The liquid formulation according to claim 1, being present in a hard gelatin capsule with banding.

4. The liquid formulation according to claim 1, wherein a solubilizer comprises a surfactant.

5. The liquid formulation according to claim 1, wherein a solubilizer comprises polysorbate 80, polyoxyethylated castor oil, or a mixture thereof.

6. The liquid formulation according to claim 1, wherein a solubilizer comprises polyoxyethylated castor oil.

7. The liquid formulation according to claim 1, wherein one or more other pharmaceutically acceptable excipients comprises one or more of polar solvents, non-polar solvents, antioxidants, glycols, and low molecular weight alcohols.

8. The liquid formulation according to claim 1, wherein one or more other pharmaceutically acceptable excipients comprises one or more of sesame oil, neutral hydrogenated vegetable oil, canola oil, corn oil, cottonseed oil, ethyl oleate, isopropyl myristate, isopropyl palmitate, caprylic triglyceride, light mineral oil, mineral oil, peanut oil, and soybean oil.

9. The liquid formulation according to claim 1, wherein one or more other pharmaceutically acceptable excipients comprises an antioxidant.

10. The liquid formulation according to claim 9, wherein an antioxidant comprises one or more of butylated hydroxytoluene, alpha-tocopherol, ascorbic acid, ascorbyl palmitate, butylated hydroxyanisole, propyl gallate, sodium ascorbate, and sodium metabisulfite.

11. The liquid formulation according to claim 1, wherein one or more other pharmaceutically acceptable excipients comprises one or more of ethanol, propanol, butanol, and mixtures thereof.

12. A process for preparing the liquid formulation of claim 1, the process comprising combining paricalcitol with at least one solubilizer, optionally together with one or more pharmaceutically acceptable excipients, and filling the liquid into a hard gelatin capsule or encapsulating to prepare a soft gelatin capsule.

13. A process for preparing the liquid formulation of claim 1, the process comprising combining paricalcitol with at least one solubilizer, optionally together with one or more pharmaceutically acceptable excipients, filling the liquid into a hard gelatin capsule, and band sealing the capsule.

14. A method for prevention and/or treatment of secondary hyperparathyroidism associated with chronic kidney disease Stages 3 and 4, or chronic kidney disease Stage 5, in patients on hemodialysis or peritoneal dialysis, using the liquid formulation according to claim 1.

15. A liquid formulation for oral administration, comprising paricalcitol, a surfactant, and one or more of polar solvents, non-polar solvents, antioxidants, glycols, and low molecular weight alcohols.

16. The liquid formulation according to claim 15, wherein a surfactant comprises polysorbate 80, polyoxyethylated castor oil, or a mixture thereof.

17. The liquid formulation according to claim 15, wherein a surfactant comprises polyoxyethylated castor oil.

18. The liquid formulation according to claim 15, comprising one or more of sesame oil, neutral hydrogenated vegetable oil, canola oil, corn oil, cottonseed oil, ethyl oleate, isopropyl myristate, isopropyl palmitate, caprylic triglyceride, light mineral oil, mineral oil, peanut oil, and soybean oil.

19. The liquid formulation according to claim 15, comprising one or more of butylated hydroxytoluene, alpha-tocopherol, ascorbic acid, ascorbyl palmitate, butylated hydroxyanisole, propyl gallate, sodium ascorbate, and sodium metabisulfite.

20. The liquid formulation according to claim 15, comprising one or more of ethanol, propanol, butanol, and mixtures thereof.
